(19) United States
(12) Patent Application Publication
(10) Pub. No.: US 2007/0128276 A1
(43) Pub. Date: Jun. 7, 2007

(54) Controlled release compositions comprising nimesulide

(75) Inventors: Rajesh Jain, New Delhi (IN); Kour Chand Jindal, New Delhi (IN); Munish Talwar, New Delhi (IN)

Correspondence Address: LADAS & PARRY, 26 WEST 61ST STREET, NEW YORK, NY, 10023, US

(73) Assignee: PANACEA BIOTEC LTD.

(21) Appl. No.: 11/545,718

(22) Filed: Oct. 10, 2006

Related U.S. Application Data
(63) Continuation-in-part of application No. 10/089,020, filed on Mar. 27, 2003.

(30) Foreign Application Priority Data
Sep. 28, 1999 (IN) INDIA 1297/DEL/99

(51) Int. Cl. A61K 9/22; A61K 31/165

(52) U.S. Cl. 424/468; 514/618

(57) ABSTRACT

A controlled release composition comprising nimesulide as an active agent formulated as a gastroretentive system, preferably as a solid oral dosage form is provided, wherein the residence time of the active agent is increased in the stomach, duodenum, jejunum or ileum. The present invention also provides process of preparing such dosage form and methods of using such dosage form compositions. The dosage form compositions are preferably administered once-a-day or twice-a-day and are particularly very useful in the prophylaxis or treatment of NSAID indicated disorder(s) such as acute painful conditions like post-operative trauma, pain associated with cancer, sports injuries, migraine headache and the like, or chronic diseases such as arthritis and the like.

### PARENT CASE TEXT

This application is continuation-in-part application of U.S. Ser. No. 10/089,020 filed on Mar. 25, 2002, which is a National Phase application of PCT International Application No. PCT/IN00/00094 filed on Sep. 27, 2000, having a priority of Sep. 28, 1999 (1297/DEL/99); the contents of which are hereby incorporated by reference into the present application.

### FIELD OF THE INVENTION

The present invention relates to a controlled release composition of nimesulide in the form of a solid oral dosage form, preferably a tablet or capsule. The present invention also provides process of preparing such dosage form and methods of using such dosage form compositions. The dosage form compositions are preferably administered once-a-day or twice-a-day and are particularly very useful in the prophylaxis or treatment of NSAID indicated disorders such as acute painful conditions like post-operative trauma, pain associated with cancer, sports injuries, migraine headache and the like or chronic diseases such as arthritis and the like.

### BACKGROUND OF THE INVENTION

Nimesulide is a nonsteroidal anti-inflammatory drug (NSAID) that also has antipyretic and analgesic properties. The compound is weakly acidic (pKa=6.5) and differs from other NSAIDs in that its chemical structure contains a sulfonanilide moeity as the acidic group. The therapeutic effects of NSAIDs are largely the result of their ability to inhibit prostaglandin synthesis via inhibition of cyclooxygenase. Unfortunately, this effect is also responsible for the inhibition of gastroprotective prostaglandins, which leads to gastrointestinal intolerance. In vitro, Nimesulide is a relatively weak inhibitor of prostaglandin synthesis and appears to exert its effects through a variety of mechanisms (Magni E.; The effect of nimesulide on prostanoid formation; Drugs 1993; 46 Suppl. 1:10-4). Indeed, the mechanism of action of this drug is more complex than previously thought and may involve interference with the production/action of mediators other than prostaglandins such as enzymes, toxic oxygen derivatives, cytokines, platelet-activating factor (PAF) and histamine.

The anti-inflammatory, analgesic and antipyretic activities of nimesulide have been demonstrated in a number of experimental models and in numerous clinical trials. Nimesulide has exhibited potency similar to or greater than that of indomethacin, diclofenac, piroxicam and ibuprofen in standard animal models of inflammation such as carrageenan-induced rat paw oedema and inflammation, ultraviolet light-induced erythema in guinea-pigs and adjuvant arthritis in rats. The analgesic potency in nimesulide was similar to that of ibuprofen and less than that of indomethacin in an acetic acid writhing test in rats, and acetic acid and acetycholine writhing tests in mice. Nimesulide has shown superior antipyretic potency to indomethacin, ibuprofen, aspirin and paracetamol (acetaminophen) in rats with yeast-induced fever.

Nimesulide is a relatively weak inhibitor of prostaglandin synthesis in vitro and appears to exert its effects through a variety of mechanisms including free-radical scavenging, effects on histamine release, the neutrophil mycloperoxidase pathway, bradykinin activity, tumour necrosis factor-α release, cartilage degradation, metalloprotease synthesis, phosphodiesterase type IV inhibition, platelet aggregation and synthesis of platelet activating factor. Animal studies have suggested that nimesulide is less ulcerogenic than aspirin, indomethacin, naproxen, piroxicam and ibuprofen. Nimesulide appears to have little effect on renal prostaglandin synthesis in rats. After oral administration of nimesulide 50 to 200 mg to healthy adult volunteers, peak serum concentrations of 1.98 to 9.85 mg/L are achieved within 1.22 to 3.17 hours. Compared with values obtained with oral drug administration, peak serum concentrations are slightly lower (2.14 to 2.32 mg/L) and are achieved more slowly (3 to 4.58 h) after rectal administration of nimesulide 100 and 200 mg. Oral drug absorption is nearly complete and concomitant administration of food may decrease the rate, but not the extent of absorption of nimesulide. The drug is extensively bound (99%) to plasma proteins and has an estimated apparent volume of distribution of 0.19 to 0.35 L/kg following oral administration.

Different dosage forms are reported for nimesulide such as tablets, granules, suppositories and suspension (Drugs 48 (3): 431-454, 1994), transdermal (U.S. Pat. No. 5,688,829) and intramuscular injection (U.S. Pat. No. 5,716,609) formulations. These reported dosage forms have to be administered twice-a-day based on biological half-life of nimesulide. The usual oral/rectal dosage of nimesulide in adults is 100 to 200 mg twice daily, orally. For treatment of chronic diseases like arthritis the twice-daily dosing regimen is difficult to comply with One approach to improve the possible non-compliance with the dosage regimen is to preferably develop controlled release dosage form for nimesulide. The once-a-day dosage form is expected to significantly increase the dosing convenience and patient compliance. Also the formulation of nimesulide as controlled release composition either for once-a-day or twice-a-day administration provides a constant release of nimesulide for extended period of time, thus providing a better efficacy in treatment of NSAID indicated disorders.

Controlled release compositions for oral use in the form of matrix type monolithic tablets, beads, capsules and coated tablets are known. However poorly soluble drugs like nimesulide are known to give erratic and variable release under in-vivo conditions from such dosage forms. One approach to formulate modified release dosage forms of NSAIDs is described in PCT publication no. WO9912524, which discloses multiple-unit compositions for administration of a therapeutically and/or prophylactically effective amount of a NSAID, said dosage form comprising at least two NSAID-containing fractions; a first NSAID-containing fraction of multiple units for quick relase of the NSAID, wherein said fraction comprises an antacid or an alkaline agent and a second NSAID-containing fraction of multiple-units in the form of coated delayed release multiple units for extended release of the NSAID substance. According to this invention, a dosage unit refers to one single unit like for e.g. a capsule or a tablet representing a plurality of individual units. But such dosage unit like tablets obtained by compression of coated multiple units causes fracturing of the coat layer, thereby causing loss of reproducibility. PCT publication bearing no. WO 95/14460 describes such compositions, which initially release a burst of a therapeutic agent and then release the agent at an essentially constant rate for extended time period. U.S. Pat. No. 5,788,987 (Busetti et al.) describes a time-specific controlled release dosage form. Such dosage forms are designed to provide delayed release of the active ingredient rather than extended release. Such formulations are not suitable for daylong management of the disease. US publication no. 20030170303 describes an orally deliverable pharmaceutical composition comprising a therapeutically effective amount of a selective cyclooxygenase-2 inhibitory drug of low water solubility and one or more pharmaceutically acceptable polymers, wherein the composition provides an in vitro sustained-release dissolution profile. U.S. Pat. No. 6,187,343 discloses a method for preparing a compound of granules of prolonged action containing 4-nitro-2-phenoxymethanesulfonanilide as active agent.

However, there exists a need to develop oral controlled release compositions comprising NSAID preferably for once-a-day prophylaxis or treatment of NSAID indicated disorders, which are effective and safe, and can be prepared in an easy and cost effective manner. A gastroretentive system with increased residence time of NSAID, such as nimesulide, in the in the desired site of absorption such as stomach, duodenum, jejunum and/or ileum would be very advantageous over all other prior arts particularly in terms of providing a better absorption of the NSAID. Such composition has not been suggested earlier in any prior art, and the present invention attempts to alleviate the limitations of the prior art by providing such novel compositions.

### SUMMARY OF THE INVENTION

It is an objective of the present invention to provide a controlled release pharmaceutical composition of nimesulide which comprises nimesulide as an active agent from 0.1% to 99% w/w of the composition, one or more release controlling materials from 0.1% to 99% w/w of the composition and one or more pharmaceutical excipients from 0.9% to 90% w/w of the composition. It is a further objective of the present invention to provide a controlled release pharmaceutical composition of nimesulide which comprises nimesulide as an active agent from 0.1% to 99% w/w of the composition, one or more release controlling materials from 0.1% to 99% w/w of the composition and one or more pharmaceutical excipients from 0.9% to 90% w/w of the composition wherein the said composition is formulated as a gastroretentive system such that the residence time of nimesulide is increased in the stomach, duodenum, jejunum and/or ileum.

It is an objective of the present invention to provide a controlled release pharmaceutical composition of nimesulide which comprises nimesulide as an active agent from 0.1% to 99% w/w of the composition, one or more release controlling materials from 0.1% to 99% w/w of the composition and one or more pharmaceutical excipients from 0.9% to 90% w/w of the composition, additionally with one or more release modifiers selected from a group comprising wetting agents, solubilizers, surfactants, plasticizers, pore formers, pH modifiers and tonicity adjusting agents, wherein the said composition is formulated as a gastroretentive system such that the residence time of nimesulide is increased in the stomach, duodenum, jejunum and/or ileum.

It is also an objective of the present invention to provide a controlled release composition of nimesulide preferably in the form of a tablet or capsule, which is formulated as a gastroretentive system wherein the residence time of nimesulide is increased in the stomach, duodenum, jejunum or ileum and wherein the gastroretention of nimesulide is achieved by mucoadhesion, floatation and/or reducing gastrointestinal motility.

It is also an objective of the invention to provide the said controlled release composition of nimesulide, wherein the said composition is formulated by compressing or compacting powder, granules, pellets, beads, shear form particles, floss, or the like, or combinations thereof. The said composition of the present invention may be filled into capsule or made into a capsule, wherein the said capsule is in the form of a hard gelatin capsule or soft gelatin capsule.

It is also an objective of the present invention to provide said controlled release composition of nimesulide, wherein the composition is in the form of a compressed or compacted multiparticulate composition comprising a blend of one or more types of particles, granules, pellets, beads, compacts, minitablets, shear form particles, floss, or the like, or combinations thereof, having different release characteristics or a multiparticulate composition made into a capsule or filled into a capsule.

It is also an objective of the present invention to provide said controlled release composition of nimesulide, wherein the controlled release composition is in the sustained release form, timed release form, pulsatile release form, prolonged release form, extended release form or delayed release form, or a combination thereof. The controlled release composition can also additionally comprise an immediate release composition.

It is also an objective of the present invention to provide said controlled release composition of nimesulide formulated as multilayered compositions like a bilayered composition, wherein one fraction is provided as an immediate release or fast release fraction providing an immediate release of the active agent and the other fraction as an extended release fraction that releases the active agent over an extended period of time.

It is another objective of the present invention to provide a process for manufacture of said controlled release pharmaceutical composition of nimesulide, which comprises of following steps:
i) treating the active agent nimesulide in an amount of from 0.1% to 99% w/w of the composition, with one or more release controlling materials in an amount of from 0.1% to 99% w/w of the composition,
ii) optionally adding one or more pharmaceutical excipients in an amount of from 0.9% to 90% w/w of the composition,
iii) formulating the material of step (ii) into a suitable composition.

It is yet another objective of the present invention to provide a method of prophylaxis or treatment of NSAID indicated disorders which comprises administrating to a patient in need thereof a pharmaceutically effective amount of the said controlled release pharmaceutical composition of nimesulide.

It is a still further objective of the present invention to provide a method of treatment of NSAID indicated disorders by administrating to a patient in need thereof a pharmaceutically effective amount of the said composition of nimesulide, wherein the NSAID indicated disorders is selected from a group comprising but not limited to pain and/or inflammation associated with osteoarthritis; dental extraction or surgery; saphenectomy or inguinal hernioplasty; haemorrhoidectomy; acute musculoskeletal injury; ear, nose or throat disorders; gynaecological disorders; cancer pain; alzheimer's disease; thrombophlebitis; urogenital disorders; bursitis or tendonitis; morning stiffness associated with rheumatoid arthritis; pain associated with fever and/or any inflammation, and the like, or a combination thereof.

The controlled release dosage form compositions of the present invention are preferably administered once-a-day or twice-a-day, which provide an extended release of nimesulide in-vivo with reproducible bioavailability. The compositions of the present invention are formulated as a gastroretentive system, which is intended to deliver nimesulide substantially at the desired site of absorption that is preferably the upper part of the gastrointestinal tract such that the residence time of the active agent is increased in the stomach, duodenum, jejunum and/or ileum. Further the release of nimesulide from such dosage forms is not affected by pH changes in the gastrointestinal environment. The compositions can be prepared in an easy and cost effective manner.

### BRIEF DESCRIPTION OF THE DRAWINGS

FIG. 1 shows the results of the fasted study.

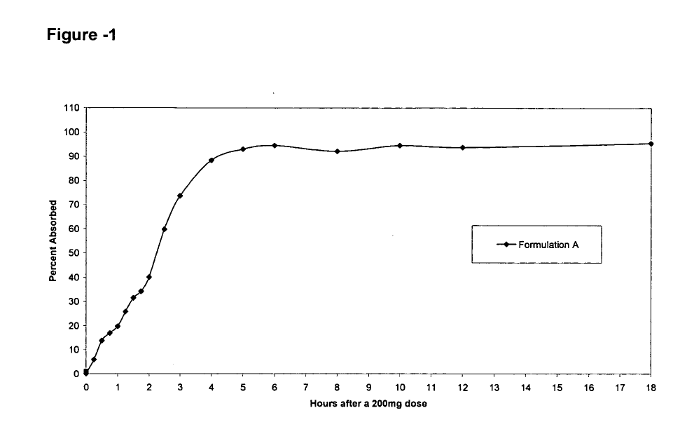

FIG. 2 shows the plot of a fed study.

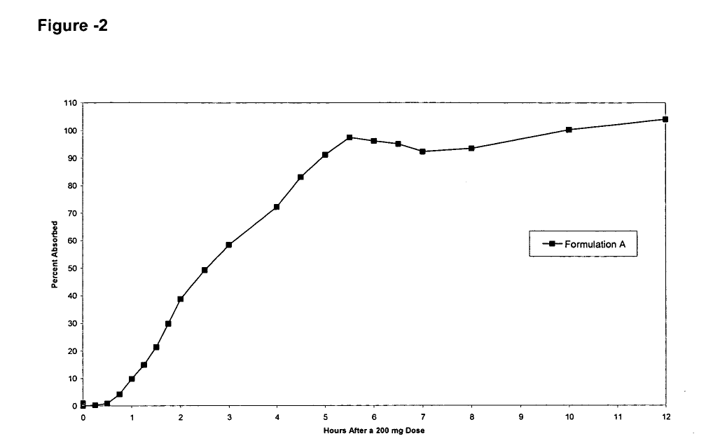

### DETAILED DESCRIPTION OF INVENTION

In accordance with the present invention there is disclosed a controlled release composition comprising nimesulide. The present invention provides a controlled release pharmaceutical composition of nimesulide which comprises nimesulide as an active agent from 0.1% to 99% w/w of the composition, one or more release controlling materials from 0.1% to 99% w/w of the composition and one or more pharmaceutical excipients from 0.9% to 90% w/w of the composition. The controlled release composition is preferably formulated as a gastroretentive system such that the residence time of nimesulide is increased in the stomach, duodenum, jejunum and/or ileum, so as to deliver nimesulide substantially at the desired site of absorption that is preferably the upper part of the gastrointestinal tract. The controlled release composition of nimesulide preferably formulated as an oral dosage form, is more preferably in the form of a tablet or capsule, which is formulated as a gastroretentive system, wherein the residence time of nimesulide is increased in the stomach, duodenum, jejunum and/or ileum and wherein the gastroretention of nimesulide is achieved by mucoadhesion, floatation and/or reducing gastrointestinal motility. In an embodiment, the release controlling material used in the composition of the present invention is a swellable polymer. In an embodiment, the controlled release composition comprises nimesulide as an active agent from 5% to 95% w/w of the composition, one or more release controlling materials from 2% to 95% w/w of the composition and one or more pharmaceutical excipients from 3% to 80% w/w of the composition.

In another embodiment, the release controlling materials of the present invention are preferably hydrophilic in nature. The release controlling materials comprise materials, which are non-toxic and pharmaceutically acceptable. These may be natural, semi-synthetic, synthetic or man-modified. Suitable materials include but not limited to cellulose and cellulose derivatives like microcrystalline cellulose, methylcellulose, ethyl cellulose, hydroxypropyl methylcellulose, hydroxypropyl cellulose, cellulose acetate phthalate, cellulose acetate, cellulose acetate butyrate, cellulose acetate propionate, cellulose acetate trimellitate, cellulose carboxymethyl ethers and their salts, hydroxypropyl methylcellulose phthalate, hydroxypropyl methylcellulose acetate succinate, polyethylene; polyquaternium-1; polyvinyl acetate (homopolymer); polyvinyl acetate phthalate; propylene glycol alginate; polyvinyl methacrylate(PVM)/methacrylic acid(MA) copolymer; polyvinyl pyrrolidone (PVP); PVP/dimethiconylacrylate/polycarbamyl/polyglycol ester; PVP/dimethylaminoethyl methacrylate copolymer; PVP/dimethylaminoethylmeth-acrylate/polycarbamyl/polyglycol ester; PVP/polycarbamyl polyglycol ester; PVP/vinyl acetate (VA) copolymer; lanolin and lanolin derivatives; glyceryl monostearate; stearic acid; paraffins; beeswax; carnauba wax; tribehenin; polyalkylene polyols like polyethylene glycols; gelatin and gelatin derivatives; alginates; carbomers; polycarbophils; methacrylic acid polymers and copolymers; carrageenans; pectins; chitosans; cyclodextrins; lecithins; natural and synthetic gums containing galactomannans like xanthan gum, tragacanth, acacia, agar, guar gum, karaya gum, locust bean gum, gum arabic, and the like, used either alone or in combination thereof.

Pharmaceutical excipients used in the composition are selected from the group of excipients generally used by persons skilled in the art e.g. carrier, filler, bulking agent, colorant, stabilizer, preservative, lubricant, glidant, chelating agent, and the like, or mixtures thereof. The pharmaceutically acceptable carrier of the present invention comprises a polymeric material selected from but not limited to the group comprising pH dependent polymers; pH independent polymers; swellable polymers; hydrophilic polymers; hydrophobic polymers and/or one or more other hydrophobic materials; ionic polymers such as sodium alginate, carbomer, calcium carboxymethylcellulose or sodium carboxymethylcellulose; non-ionic polymers such as hydroxypropyl methylcellulose; synthetic or natural polysaccharide selected from the group comprising alkylcelluloses, hydroxyalkylcelluloses, cellulose ethers, cellulose esters, nitrocelluloses, dextrin, agar, carrageenan, pectin, furcellaran, starch and starch derivative, and mixtures thereof. The polymeric material used in the present invention is selected from but not limited to a group comprising cellulosic polymer, methacrylate polymer, PVP, alginate, PVP-PVA copolymer, ethylcellulose, cellulose acetate, cellulose propionate (lower, medium or higher molecular weight), cellulose acetate propionate, cellulose acetate butyrate, cellulose acetate phthalate, cellulose triacetate, poly(alkyl methacrylate), poly(isodecyl methacrylate), poly(lauryl methacrylate), poly(phenyl methacrylate), poly(alkyl acrylate), poly(octadecyl acrylate), poly(alkylene), poly(alkylene oxide), poly(alkylene terephthalate), poly(vinyl isobutyl ether), poly(vinyl acetate), poly(vinyl chloride) and polyurethane or a mixture thereof; used either alone or in combination thereof.

In an embodiment of the present invention, the pharmaceutically acceptable excipients are selected from a group comprising but not limited to carriers, diluents, binders, disintegrants, colorants, lubricants, antiadherants, plasticizers, coating agents, opacifiers, antioxidants, stabilizers, preservatives, surfactants, hydrophilic polymers, solubility enhancing agents, osmotic agents, and the like used either alone or in combination thereof. In another embodiment, the composition of the present invention further comprises one or more release modifiers selected from a group comprising but not limited to wetting agents, solubilizers, surfactants, plasticizers, pore formers, pH modifiers and tonicity adjusting agents, or combination thereof.

In a preferred embodiment of the invention, controlled release of nimesulide from the formulation may be achieved by designing a gastroretentive system. Nimesulide is absorbed mainly from the stomach and proximal part of small intestine that is the upper part of the gastrointestinal tract. It is not well absorbed from distal part of small intestine and large intestine; instead such administration leads to loss of bioavailability. Active agents having such characteristics require gastroretentive systems wherein residence time of drug is increased in stomach, duodenum, jejunum or ileum. Gastroretentive characteristics may be incorporated into dosage form/drug delivery system by techniques such as treating active agent with polymers having specific affinity to bind with gastric mucosa, reducing specific gravity of the dosage form leading to floatation, increasing size of the dosage form such that it is greater than the pyloric diameter, and/or using chemicals which delay gastric emptying, and the like, or a combination of more than one such techniques. In an embodiment, the gastroretentivity of the dosage form composition might also be achieved by delaying the gastric emptying time such as by administration of food.

In a more preferred embodiment, the controlled release pharmaceutical composition of the present invention is formulated as a gastroretentive system, wherein the residence time of nimesulide is increased in the stomach, duodenum, jejunum or ileum. In a further embodiment, the gastroretention of nimesulide is achieved by mucoadhesion, floatation and/or reducing gastrointestinal motility. The mucoadhesion is achieved by treating nimesulide with polymers having affinity for gastrointestinal mucosa selected from a group comprising but not limited to polycarbophils, carbomers, alginates, cellulose and cellulose derivatives, chitosan, gums and lectins, or mixtures thereof. Floatation is achieved by adding to the composition one or more gas-generating materials comprising but not limited to sodium bicarbonate, sodium carbonate, calcium carbonate, potassium carbonate or the like, alone or in combination with one or more acidic substances comprising but not limited to hydrochloric acid, citric acid, fumaric acid, malic acid, maleic acid, ascorbic acid or tartaric acid, or mixtures thereof. The gastrointestinal motility is reduced by adding materials selected from a group comprising but not limited to fats, fatty acids and transesterification products of fats and fatty acids with polyols, or mixtures thereof.

In an embodiment of the present invention, the controlled release composition of nimesulide is formulated in the sustained release form, timed release form, pulsatile release form, prolonged release form, extended release form or delayed release form, or a combination thereof. In a preferred embodiment, the controlled release form is in the form of a combination of immediate release form and extended release form.

A randomized open label balanced single dose study was carried out using the compositions of the present invention i.e. Nimesulide 200 mg extended release (ER) tablets (compositions of Example-1 described herein, referred to as ‘Formulation-A’) on healthy male adult human subjects under both fasted conditions and fed conditions. It was found that food significantly increased the absorption of nimesulide from the ER tablet, as seen in an increase of the relative extent of absorption from ˜50% in the fasted study to almost about 80% when dosed following consumption of food with high fat content.

Wagner-Nelson absorption plots were constructed for the single 200 mg doses of Nimesulide ER tablets for each subject in the fasted and fed studies. These plots were normalized to the asymptotic Wagner-Nelson function value of compensate for extent of absorption differences. This enabled an evaluation of the rate of drug absorption of the compositions studied. The plots also provided information on the fraction of drug absorbed at different time intervals besides the fraction of drug absorbed and unabsorbed ultimately. The plot for the fasted study is presented as FIG.-1 and the plot for the fed study is presented as FIG.-2. The study results indicate that the absorption process takes place for at least about 4 hours and appears to be substantially complete for the ER tablets following a fasted dose. For the ER tablets (compositions of Example-1 described herein, referred to as ‘Formulation-A’) given after a meal, however, absorption continues for at least about 6 hours. From the absorption plots, it appears that the median times to achieve at least about 90% absorption were about 4-8 hours for the single 200 mg dose of ‘Formulation-A’. These findings are consistent with the literature available on nimesulide which indicates that its absorption takes place primarily in the upper gastrointestinal tract, with little occurring from the colon. The average transit time across the upper gastrointestinal tissues, wherein nimesulide absorption occurs preferably, is typically between 3.5-5 hours for administration in the fasted state. This transit time includes both the time needed for gastric emptying and the time it takes for nimesulide to move through the small bowel between the pyloric sphincter to ascending colon.

In a preferred embodiment, the Nimesulide ER composition of the present invention is designed to release most of the drug content in the first 6-8 hours. If the fasted absorption window is between 3.5-5 hours (stomach to ascending colon), then it is logical that the extent of nimesulide absorption for the ER tablets following a fasted dose would be only about 50% as observed in this study. The increase in transit time through the upper gastrointestinal tract, a known effect of food, provides an absorption window for the Nimesulide ER product that is significantly increased beyond that which occurs following a fasted administration of the said product. Consequently, the extent of nimesulide absorption from the ER tablet given after a meal is greater than that when it is given in the fasted state, since the longer absorption window occurs when dosing after a meal. In an embodiment, the mean retention time (MRT) of the controlled release composition comprising Nimesulide ranges from about 5-10 hours in upper part of the gastrointestinal tract that is a preferred site of absorption wherein the drug is substantially completely released and wherefrom the drug is substantially absorbed. Further, the study results also indicate that there is no dose dumping in the presence of food. In the fed state, the bioavailability of nimesulide from the dosage form is enhanced compared to the fasted state. It could also be inferred from the study that food increases the extent of absorption, but does not significantly affect the kinetics of absorption.

In another embodiment, the dosage form of the present invention additionally comprises at least one surfactant selected from but not limited to a group comprising anionic surfactants, cationic surfactants, non-ionic surfactants, zwitterionic surfactants, or mixtures thereof. In yet another embodiment, the dosage form of the present invention additionally comprises at least one complexing agent such as cyclodextrin selected from a group comprising but not limited to alpha-cyclodextrin, beta-cyclodextrin, betahydroxy-cyclodextrin, gamma-cyclodextrin, and hydroxypropyl cyclodextrin, or the like.

Preferably the composition also comprises release modifiers. Such release modifiers are selected from but not limited to a group comprising wetting agents, solubilizers, surfactants, plasticizers, solvents, pore formers, pH modifiers, tonicity adjusting agents, and the like, or mixtures thereof. Suitable examples of such ingredients include reaction products of natural and hydrogenated vegetable oils and ethylene glycol e.g. polyoxyethylene glycolated natural or hydrogenated castor oil such as CREMOPHOR®. Other suitable products include polyoxyethylene sorbitan fatty acid esters e.g. TWEEN®; polyoxyethylene fatty acid esters e.g. MYRJ® and CETIOL® HE; polyoxyethylene polyoxypropylene copolymers e.g. PLURONIC® and polyoxyethylene polyoxypropylene block copolymers e.g. POLOXAMER®; dioctylsodiumsulfosuccinate, sodium lauryl sulphate; propylene glycol mono- and di-fatty acid esters e.g. MIGLYOL® 840; bile salts such as alkali metals salts e.g. sodium taurocholate; polyethylene glycols, propylene glycol, triacetin, diacetin, diethyl phthalate, dibutyl phthalate, castor oil, triethyl citrate dibutyl sebacate, sodium chloride, potassium chloride, lactose, mannitol, sucrose, sorbitol, sodium hydroxide, potassium hydroxide, sodium bicarbonate, sodium citrate, citric acid, hydrochloric acid, lactic acid, tartaric acid, malic acid, and the like, or mixtures thereof.

In an embodiment, the composition is formulated in a bimodal release form such as an immediate release form to provide an initial loading dose of the active agent and a sustained release form to provide a release of the active agent for an extended duration. In another embodiment, the composition comprises at least two fractions wherein one fraction provides an immediate release of nimesulide and at least one other fraction provides an extended release of nimesulide, optionally with one or more other fraction(s). When the composition is formulated as a tablet, it is formed by compacting or compressing the said fractions. When formulated as a capsule, the fractions are mixed and filled into suitable-sized capsules or compressed/compacted and then filled into capsules. The capsule may be in the form of hard gelatin capsule or soft gelatin capsule. In another embodiment, the composition is formulated as a multilayer tablet composition. Preferably the tablet composition is formulated as bilayered composition. More preferably, the bilayered composition comprises one layer as an immediate release or fast release layer providing an immediate release of nimesulide and the other layer as an extended release layer that releases nimesulide over extended periods of time.

In an embodiment, the composition of the present invention comprises at least two fractions wherein one fraction is an immediate release or fast release fraction providing an immediate release of the active agent and the other fraction is an extended release fraction that releases the active agent over extended periods of time.

In another embodiment, the present invention provides a controlled release composition of nimesulide, wherein the said composition is formulated by compressing or compacting powder, granules, pellets, beads, compacts, shear form particles, floss, or the like, or combinations thereof, or formulating the composition into a tablet or minitablet or capsule or filling the composition into a capsule. In another embodiment, the composition of the present invention provides a controlled release composition of nimesulide, wherein the composition is in the form of a compressed or compacted multiparticulate composition comprising a blend of one or more types of particles, granules, pellets, beads, compacts, shear form particles, floss, or the like, or combinations thereof, having different release characteristics. In an embodiment, the composition in the form of a tablet is prepared by either direct compression, dry compression (slugging), or by granulation. The granulation technique is either aqueous or non-aqueous. In another embodiment, the composition of the present invention filled into a capsule is in the form of a multiparticulate composition comprising a blend of one or more types of particles, pellets or mini-tablets having different release characteristics. In an embodiment, the composition of the present invention filled into a capsule is formulated in the form of tablets. The tablets can be prepared by either direct compression, dry compression (slugging), or by granulation. The granulation technique is either aqueous or non-aqueous. In a further embodiment, the multiparticulate composition is in the form of a compressed or compacted minitablet or tablet or a hard gelatin capsule or a soft gelatin capsule.

In a further embodiment, the composition of the present invention is formulated as a matrix type controlled release dosage form or as an extended release membrane diffusion controlled dosage form or as a site-targeted device. The extended release membrane diffusion controlled dosage form or site-targeted device may comprise one or more excipients selected from but not limited to a group comprising diluents such as lactose, mannitol, microcrystalline cellulose, and the like; mucoadhesive polymers such as hydroxypropyl methylcellulose, polycarbophil, polyoxyethylene oxide and the like, or mixtures thereof; binders such as povidone, povidone/vinyl acetate copolymer, starch, hydroxypropyl cellulose, and the like, or mixtures thereof; methacrylates and methacrylic acid copolymer such as various grades of Eudragits® and the like, or mixtures thereof; cellulosic polymers such as hydroxypropyl methylcellulose, ethyl cellulose, hydroxypropyl cellulose, and the like, or mixtures thereof; plasticizer such as triethyl citrate, polyethylene glycol, and the like, or mixtures thereof; channel forming agents such as polyethylene glycol, lactose, dextrose, maltitol, and the like, or mixtures thereof; lubricants such as talc, magnesium stearate, and the like, or mixtures thereof; organic solvents such as isopropyl alcohol and/or dichloromethane, or mixtures thereof. The polymers are useful as a granulating aid and/or as a coating agent, preferably dissolved or dispersed in an aqueous, non-aqueous or a hydro-alcoholic medium.

In a preferred embodiment of the invention, the composition comprises one or more fractions wherein nimesulide may be present in one or all fractions. In a further embodiment, the composition is in the form of multilayered tablets like bilayer tablets or minitablets comprising a fast release fraction which gives fast release of the drug and an extended release fraction which gives extended release of the drug, wherein the active agent may be present in one or both fractions. In a still further embodiment, the composition is in the form of bilayer tablet or minitablet comprising a fast release layer which gives fast release of the active agent and an extended release layer which gives extended release of the active agent, wherein the active agent may be present in one or both layers. The bilayer tablets or minitablets may be coated or uncoated. The tablet or minitablet is preferably coated with one or more coating layers by a coating material comprising at least a film forming agent, a channel forming agent, a plasticizer, and an aqueous/non-aqueous solvent. The coating may also be in the form of a semi-permeable type membrane. Further, the semi-permeable coat may have an orifice drilled through it on the drug layer side to provide passage for constant release of drug. In another aspect of the invention, the coating may be of microporous type through which the release of the active agent takes place preferably at constant rate. The first layer providing fast release of the active agent comprises one or more materials such as binders, disintegrants, fillers, rapidly soluble/dispersible excipients, wetting agents, and the like or mixtures thereof. The second layer providing extended release of the active agent comprises one or more materials such as rate controlling polymers, binders, wetting agents, disintegrants, fillers, and the like or mixtures thereof. In another embodiment the said tablet or minitablet composition is filled into capsule comprising at least two fractions, one fraction being in the fast release or immediate release form and the other fraction being in the extended release form.

In another embodiment, the composition of the present invention is formulated as an osmotically controlled constant release type device or as pH dependent delayed release type or a pulsatile release type or as hydrodynamically balanced system. In an embodiment, the hydrodynamically balanced system comprises one or more excipients selected form a group comprising polymers such as carbomers such as Carbopol®934P and Carbopol®974; celluloses such as hydroxypropyl cellulose; gums such as xanthan gum; alginic acid or alginates such as sodium alginate; gas-generating materials such as sodium bicarbonate; diluents such as lactose; binders such as polyvinyl pyrrolidone; granulating aids such as isopropyl alcohol; lubricants such as magnesium stearate; antiadherants such as colloidal silicon dioxide or talc; and the like or mixtures thereof. In another embodiment, the pH dependent delayed release type composition comprises a pH dependent polymer, which releases the active agent at a specific site of the GIT. In a further embodiment, the osmotically controlled constant release type device comprises two fractions wherein one fraction comprises the active agent along with one or more release controlling materials optionally with a tonicity adjusting agent such as sodium chloride and the other fraction comprises one or more swellable mucoadhesive polymer optionally along with a tonicity adjusting agent. In another embodiment of the present invention, the composition is formulated as effervescent or dispersible system. In another embodiment, the tablet composition is formulated as gastric mucoadhesive controlled release monolithic or layered tablets.

In a further embodiment, the composition comprises a permeation enhancer, selected from but not limited to a group comprising Vitamin E tocopheryl propylene glycol succinate (Vitamin E TPGS), piperine, a lipid, or a surfactant, or mixtures thereof.

In another embodiment, the composition is formulated as a film coated or enteric coated dosage form. The coating material used for film coating and/or enteric coating is selected from but not limited to the conventionally used materials such as cellulosic polymers, methacrylic acid polymers and copolymers, or the like or mixtures thereof. Other conventionally used excipients for the coating includes but not limited to plasticizers, solvents, lubricants, surfactants, and the like or mixtures thereof. In another aspect of the present invention, the composition of the present invention comprises nimesulide in micronized form, preferably having average particle size below 20 microns, more preferably below 5 microns.

In a further embodiment of the present invention is provided a process of manufacturing the said controlled release composition. In an embodiment, the process for the manufacture of controlled release pharmaceutical composition of nimesulide comprises the following steps:
i) treating the active agent nimesulide in an amount of from 0.1% to 99% w/w of the composition, with one or more release controlling materials in an amount of from 0.1% to 99% w/w of the composition,
ii) optionally adding one or more pharmaceutical excipients in an amount of from 0.9% to 90% w/w of the composition,
iii) formulating the material of step (ii) into a suitable dosage form.

In yet another embodiment is provided a method of prophylaxis or treatment of NSAID indicated disorders, which comprises administrating to a patient in need thereof a pharmaceutically effective amount of the composition of the present invention. The NSAID indicated disorders is selected from but not limited to a group comprising pain and/or inflammation associated with osteoarthritis; dental extraction or surgery; saphenectomy or inguinal hernioplasty; haemorrhoidectomy; acute musculoskeletal injury; ear, nose or throat disorders; gynaecological disorders; cancer pain; alzheimer's disease; thrombophlebitis; urogenital disorders; bursitis or tendonitis; morning stiffness associated with rheumatoid arthritis, pain associated with fever and/or inflammation, and the like, or a combination thereof, or any other disorder known to art, wherein nimesulide is indicated for treatment or prophylaxis.

In a further embodiment, the compositions of the present invention comprising nimesulide may be combined with one or more suitable preferably long-acting active agent(s). The other active agent may be present in an immediate release form or controlled release form or a combination of both forms. Suitable active agents that can be used along with nimesulide is selected from but not limited to a group comprising antihistaminics e.g. cetirizine; antispasmodics e.g. pitofenone, hyoscine hydrobromide; antiasthmatics e.g. ketotifen, salbutamol; antipyretics such as paracetamol, aspirin, and the like.

The foregoing examples are illustrative embodiments of the invention and are merely exemplary. A person skilled in the art may make variations and modifications without deviating from the spirit and scope of the invention. All such modifications and variations are intended to be included within the scope of the invention.

### EXAMPLES

### Example-1

A) Immediate Release Layer

| S. No. | Ingredient | Quantity (mg/tablet) |
|---|---|---|
| 1. | Nimesulide | 50.00 |
| 2. | Lactose | 86.53 |
| 3. | Croscarmellose sodium | 3.75 |
| 4. | Colloidal silicon dioxide | 3.00 |
| 5. | Ferric oxide red | 0.473 |
| 6. | Starch | 19.55 |
| 7. | Hydrochloric acid | q.s. |
| 8. | Docusate sodium | 3.40 |
| 9. | Povidone K-30 | 3.00 |
| 10. | Polysorbate-80 | 0.50 |
| 11. | Purified water | q.s. |
| 12. | Colloidal silicon dioxide | 2.50 |
| 13. | Povidone K-30 | 1.25 |
| 14. | Magnesium stearate | 0.80 |
| 15. | Croscarmellose sodium | 7.25 |

Procedure:
i) Blend 1, 2, 3 and 4 and pass them through a sieve of mesh size 30.
ii) Pass 5 and 6 through a sieve of mesh size 100 and blend with step (i).
iii) Dissolve 7, 8, 9 and 10 in 11.
iv) Granulate the blend of (ii) with solution of step (iii).
v) Dry the granules of step (iv) and pass them through a sieve of mesh size 20.
vi) Pass 12, 13, 14 and 15 through a sieve of mesh size 40.
vii) Blend the granules step (v) with the ingredients of step (vi).

B) Extended Release Layer

| | | Quantity |
|---|---|---|
| S. No. | Ingredient | (mg/tablet) |
| 1. | Nimesulide | 150.00 |
| 2. | Lactose | 69.75 |
| 3. | Docusate sodium | 3.00 |
| 4. | Povidone K-30 | 3.00 |
| 5. | Purified water | q.s. |
| 6. | Hydroxypropyl methylcellulose | 52.50 |
| | (high viscosity grade) | |
| 7. | Colloidal silicon dioxide | 1.50 |
| 8. | Magnesium stearate | 1.50 |
| 9. | Sodium lauryl sulphate | 0.75 |
| 10. | Colloidal silicon dioxide | 1.50 |
| 11. | Magnesium stearate | 1.50 |
| 12. | Povidone K-30 | 3.00 |

Procedure:
i) Sift 1 and 2 through a sieve of mesh size 30 and mix.
ii) Dissolve 3 and 4 in 5 to obtain homogeneous solution.
iii) Granulate material of step (i) with material of step (ii) and dry the granules and pass through a sieve of mesh size 20.
iv) Sift ingredients 6, 7, 8 and 9 together through sieve of mesh size 40 and mix with the dried granules of step (iii).
v) Compact the bulk of step (iv) and then size them through mesh size 22.
vi) Sift 10, 11 and 12 through mesh size 40.
vii) Lubricate the bulk of step (v) with the ingredients of step (vi).

Compress the material obtained in step A (vii) and the material obtained in step (B) (vii) into bilayer tablets.

### Example-2

A) Immediate Release Layer

| S. No. | Ingredient | Quantity (mg/tablet) |
|---|---|---|
| 1. | Nimesulide | 50.00 |
| 2. | Sodium lauryl sulphate | 1.50 |
| 3. | Lactose | 93.73 |
| 4. | Croscarmellose sodium | 3.75 |
| 5. | Starch | 19.55 |
| 6. | Ferric oxide red | 0.473 |
| 7. | Polyvinylpyrrolidone (Povidone K-30) | 1.50 |
| 8. | Purified water | q.s. |
| 9. | Magnesium stearate | 0.50 |
| 10. | Croscarmellose sodium | 7.25 |
| 11. | Colloidal silicon dioxide | 2.50 |
| 12. | Povidone K-30 | 1.25 |

Procedure:
i) Co-mill 1 and 2.
ii) Sift 3 and 4 through a sieve of mesh size 30.
iii) Sift 5 and 6 through a sieve of mesh size 100.
iv) Blend materials of step (i), (ii) and (iii) together.
v) Dissolve 7 in 8.
vi) Granulate the blend of step (iv) with solution of step (v).
vii) Dry the granules of step (vi) and pass them through a sieve of mesh size 20.
viii) Pass 9, 10, 11 and 12 through a sieve of mesh size 40.
ix) Blend the granules of step (vii) with the ingredients of step (viii).

B) Extended Release Layer

| | | Quantity |
|---|---|---|
| S. No. | Ingredient | (mg/tablet) |
| 1. | Nimesulide | 150.00 |
| 2. | Lactose | 70.75 |
| 3. | Docusate sodium | 3.00 |
| 4. | Povidone K-30 | 3.00 |
| 5. | Purified water | q.s. |
| 6. | Hydroxypropyl methylcellulose | 52.50 |
| | (high viscosity grade) | |
| 7. | Colloidal silicon dioxide | 1.50 |
| 8. | Magnesium stearate | 1.50 |
| 9. | Sodium lauryl sulphate | 0.75 |
| 10. | Magnesium stearate | 0.50 |
| 11. | Colloidal silicon dioxide | 1.50 |
| 12. | Povidone K-30 | 3.00 |

Procedure:
i) Sift 1 and 2 through a sieve of mesh size 30 and mix.
ii) Dissolve 3 and 4 in 5.
iii) Granulate material of step (i) with the binder solution of step (ii).
iv) Dry the granules and pass through a sieve of mesh size 20.
v) Sift ingredients 6, 7, 8 and 9 through mesh size 40 together and mix with the dried granules of step (iv).
vi) Compact the ingredients of step (v) and size them through mesh size 22.
vii) Sift ingredients 10, 11 and 12 through mesh size 40.
viii) Lubricate the ingredients of step (vi) with the ingredients of (vii).

Compress the material obtained in step (A) (ix) and the material obtained in step (B) (viii) into bilayer tablets.

### Example-3

| | | Quantity |
|---|---|---|
| S. No. | Ingredient | (mg/capsule) |
| 1. | Nimesulide (micronized) | 200.0 |
| 2. | Lactose | 66.0 |
| 3. | Hydroxypropyl methylcellulose | 70.0 |
| | (high viscosity grade) | |
| 4. | Colloidal silicon dioxide | 10.0 |
| 5. | Magnesium Stearate | 0.5 |
| 6. | Purified Talc | 3.5 |

Procedure:
i) Compact the ingredients 1, 2, 3, 4 and 5 together after sifting them through a sieve of mesh size 30 (BSS). Size them mixture through mesh size 22.
ii) Pass 6 through sieve of mesh size 40.
iii) Mix the material of step (i) with the material of step (ii).
iv) Fill the material of step (iii) into hard gelatin capsules.

### Example-4

| | | Quantity |
|---|---|---|
| S. No. | Ingredient | (mg/tablet) |
| 1. | Nimesulide | 100.0 |
| 2. | Microcrystalline cellulose | 80.0 |
| 3. | Lactose | 90.0 |
| 4. | Maize starch | 10.0 |
| 5. | Purified water | q.s. |
| 6. | Purified talc | 3.5 |
| 7. | Ethyl cellulose (as aqueous | 10.0 |
| | dispersion) | |
| 8. | Purified water | q.s. |

Procedure:
i) Sift 1, 2 and 3 through mesh size 30.
ii) Prepare binder paste of 4 in 5.
iii) Granulate ingredients of step (i) with the material in step (ii).
iv) Dry the granules of step (iii) and sift through mesh size 22.
v) Lubricate material of step (iv) with 6 passed through mesh size 40
vi) Compress material of step (v) into tablets.
vii) Coat the tablets of step (vi) with the solution of 7 in 8.

### Example-5

| S. No. | Ingredient | Quantity (mg/tablet) |
|---|---|---|
| 1. | Non-Pareil beads | 347.0 |
| 2. | Nimesulide | 200.0 |
| 3. | Mannitol | 30.0 |
| 4. | Lactose | 30.0 |
| 5. | Polyvinyl pyrrolidone | 20.0 |
| 6. | Purified water | q.s. |
| 7. | Purified talc | 15.0 |
| 8. | Ethyl cellulose | 7.0 |
| 9. | Diethyl phthalate | 1.4 |

Procedure:
i) Coat 1 with dispersion of 2, 3, 4 and 5 in 6 in a conventional or fluidized bed coater.
ii) Dust 7 onto the beads of step (i) to avoid any sticking or lump formation.
iii) Compress the material of step (ii) into tablets.
iv) Coat tablets of step (iii) with solution of 8 in 9.

### Example-6

| | | Quantity |
|---|---|---|
| S. No. | Ingredient | (mg/tablet) |
| 1. | Nimesulide | 200.00 |
| 2. | Lactose | 93.00 |
| 3. | Docusate sodium | 4.0 |
| 4. | Povidone K-30 | 4.0 |
| 5. | Purified water | q.s. |
| 6. | Hydroxypropyl methylcellulose | 70.00 |
| | (high viscosity grade) | |
| 7. | Colloidal silicon dioxide | 2.0 |
| 8. | Magnesium stearate | 2.0 |
| 9. | Sodium lauryl sulphate | 1.0 |
| 10. | Magnesium stearate | 2.0 |
| 11. | Colloidal silicon dioxide | 2.0 |
| 12. | Povidone K-30 | 4.0 |

Procedure:
i) Sift 1 and 2 through a sieve of mesh size 40 and mix.
ii) Dissolve 3 and 4 in 5.
iii) Granulate material of step (i) with the binder solution of step (ii).
iv) Dry the granules and pass through a sieve of mesh size 20.
v) Sift ingredients 6, 7, 8 and 9 through mesh size 40 together and mix with the dried granules of step (iv).
vi) Compact the ingredients of step (v) and size them through mesh size 22.
vii) Sift ingredients 10, 11 and 12 through mesh size 40.
viii) Lubricate the materials of step (vi) with the material of (vii).
ix) Compress the material of step (viii) into tablet.

### Example-7

| S. No. | Ingredient | Quantity/tablet (mg) |
|---|---|---|
| 1. | Nimesulide | 50.0 |
| 2. | Mannitol | 80.0 |
| 3. | Sodium starch glycollate | 5.0 |
| 4. | Colloidal silicon dioxide | 3.0 |
| 5. | Maize starch | 10.0 |
| 6. | Povidone K-30 | 3.0 |
| 7. | Polysorbate 80 | 1.0 |
| 8. | Purified water | q.s. (lost in |
| | | processing) |
| 9. | Magnesium stearate | 1.0 |
| 10. | Croscarmellose sodium | 8.0 |

Procedure:
i) Mix 1 to 5 together and sift through a sieve of mesh size 30.
ii) Dissolve 6 & 7 in 8 to obtain a homogeneous solution.
iii) Granulate the material of step (i) with the material of step (ii) followed by drying and sifting through a sieve of mesh size 16.
iv) Sift 9 & 10 through a sieve of mesh size 40.
v) Mix the material of step (iv) with the material of step (iii).

B) Extended Release Fraction

| S. No. | Ingredient | Quantity/tablet (mg) |
|---|---|---|
| 1. | Nimesulide | 150.0 |
| 2. | Lactose | 40.0 |
| 3. | Methacrylate polymer | 60.0 |
| 4. | Docusate sodium | 3.0 |
| 5. | Purified water | q.s. (lost in |
| | | processing) |
| 6. | Hydroxypropyl methylcellulose | 2.5 |
| 7. | Colloidal silicon dioxide | 3.5 |
| 8. | Magnesium stearate | 2.0 |

Procedure:
i) Mix 1 to 3 together and sift through a sieve of mesh size 30.
ii) Dissolve 4 in 5 to obtain a homogeneous dispersion.
iii) Granulate the material of step (i) with the material of step (ii) followed by drying and sifting through a sieve of mesh size 24.
iv) Sift 6, 7 & 8 through a sieve of mesh size 40.
v) Mix the material of step (iv) with the material of step (iii).

Mixed together and compress the material obtained in step (A) (v) and the material obtained in step (B) (v) into tablets.

### Example-8

A) Nimesulide Immediate Release Fraction

| S. No. | Ingredient | Quantity/tablet (mg) |
|---|---|---|
| 1. | Nimesulide | 50.0 |
| 2. | Mannitol | 6.5 |
| 3. | Sodium starch glycollate | 6.0 |
| 4. | Maize starch | 5.0 |
| 5. | Polysorbate 80 | 1.0 |
| 6. | Povidone (K-30) | 2.0 |
| 7. | Purified water | q.s. |
| 8. | Magnesium stearate | 1.5 |
| 9. | Colloidal silicon dioxide | 7.0 |

Procedure:
i) Mix 1 to 4 together and sift through a sieve of mesh size 30.
ii) Dissolve 5 and 6 in 7.
iii) Granulate the material of step (i) with the material of step (ii) and dry the granules.
iv) Pass the dried granules of step (iii) through mesh size 22.
v) Sift ingredients 8 and 9 through mesh size 40.
vi) Lubricate the material of step (iv) with the material of step (v).

B) Nimesulide Delayed Release Fraction

| S. No. | Ingredient | Quantity/tablet (mg) |
|---|---|---|
| 1. | Nimesulide | 50.0 |
| 2. | Lactose | 6.5 |
| 3. | Docusate sodium | 2.0 |
| 4. | Povidone K-30 | 3.0 |
| 5. | Purified water | q.s. |
| 6. | Colloidal silicon dioxide | 3.0 |
| 7. | Magnesium stearate | 3.0 |
| 8. | Methacrylate polymer | 5.5 |
| 9. | Triethyl citrate | 1.5 |
| 10. | Isopropyl alcohol | q.s. (lost in |
| | | processing) |
| 11. | Methylene chloride | q.s. (lost in |
| | | processing) |

Procedure:
i) Mix 1 and 2 together and sift through a sieve of mesh size 30.
ii) Dissolve 3 and 4 in 5.
iii) Granulate the material of step (i) with the material of step (ii) and dry the granules and pass through mesh size 16.
iv) Sift ingredients 6 and 7 through a sieve of mesh size 40.
v) Mix the material of step (iii) with the material of step (iv).
vi) Disperse ingredients 7 & 8 in a mixture of 9, 10 and 11 and mix.
vii) Coat the material of step (v) with the material of step (vi).

C) Sustained Release Fraction

| S. No. | Ingredient | Quantity/tablet (mg) |
|---|---|---|
| 1. | Nimesulide | 100.00 |
| 2. | Lactose monohydrate | 10.0 |
| 3. | Sodium carboxymethylcellulose | 7.5 |
| 4. | Docusate sodium | 3.00 |
| 5. | Povidone (K-30) | 3.00 |
| 6. | Purified water | q.s. (lost in |
| | | processing) |
| 7. | Colloidal silicon dioxide | 3.00 |
| 8. | Magnesium stearate | 3.00 |

Procedure:
i) Mix 1 to 3 together and sift through a sieve of mesh size 30.
ii) Dissolve ingredients 4 & 5 in 6 to obtain a homogeneous dispersion.
iii) Granulate the material of step (i) with the material of step (ii) followed by drying and sifting through a sieve of mesh size 18.
iv) Sift ingredients 7 & 8 through a sieve of mesh size 40.
v) Mix the material of step (iv) with the material of step (iii).

Mix together and compress the material of step (A) (vi), step (B) (vii) and step (C) (v) into tablets.

### Example-9

| S. No. | Ingredient | Quantity (mg/tablet) |
|---|---|---|
| 1. | Nimesulide (micronized) | 200.0 |
| 2. | Microcrystalline cellulose | 88.4 |
| 3. | Lactose | 70.0 |
| 4. | Polyvinyl pyrrolidone | 7.0 |
| 5. | Magnesium stearate | 3.9 |
| 6. | Ethyl cellulose | 20.0 |
| 7. | Polyethylene glycol | 0.7 |
| 8. | Alcohol:Dichloromethane (1:2) | q.s. (Lost in |
| | | processing) |

Procedure:
i) Blend ingredients 1, 2, 3, 4 and 5 and compress into tablets.
ii) Coat the tablets of step (i) with dispersion of 6 and 7 in 8.

### Example-10

| S. No. | Ingredient | Quantity (mg/tablet) |
|---|---|---|
| 1. | Nimesulide | 100.0 |
| 2. | Microcrystalline cellulose | 150.0 |
| 3. | Lactose | 176.0 |
| 4. | Polyoxyl 40 hydrogenated castor oil | 7.0 |
| 5. | Polyvinyl pyrrolidone | 10.0 |
| 6. | Magnesium stearate | 3.5 |
| 7. | Purified talc | 3.5 |
| 8. | Cellulose acetate phthalate | 28.0 |
| 9. | Diethyl phthalate | 2.0 |
| 10. | Purified water | q.s. (Lost in |
| | | processing) |
| 11. | Alcohol:Dichloromethane (1:2) | q.s. (Lost in |
| | | processing) |

Procedure:
i) Granulate the blend of ingredients 1, 2 and 3 with solution of 4 and 5 in 10 and dry the granules.
ii) Blend the dried granules of step (i) with ingredients 6 and 7 and compress into tablets.
iii) Coat the tablets of step (ii) with the dispersion of 8 and 9 in 11.

### Example-11

| | | Quantity | | |
|---|---|---|---|---|
| | | (mg/tablet) | | |
| S. No. | Ingredient | I | II | III |
| 1. | Nimesulide (micronized) | 100.0 | 50.0 | 50.0 |
| 2. | Microcrystalline cellulose | 200.0 | 200.0 | 200.0 |
| 3. | Lactose | 50.0 | 42.0 | 35.0 |
| 4. | Polyvinyl pyrrolidone | 10.0 | 10.0 | 10.0 |
| 5. | Water | q.s. | q.s. | q.s. |
| 6. | Ammonio methacrylate copolymer Type B | 10.0 | 18.0 | 25.0 |
| 7. | Diacetin | 0.5 | 0.5 | 0.5 |
| 8. | Water:Acetone (1:9) | q.s. | q.s. | q.s. |

Procedure:
i) Prepare 3 types of beads coated with different amounts of 6 to give a timed release profile of nimesulide.
ii) Prepare the beads by blending and spheronizing ingredients 1, 2 and 3 using aqueous solution of 4.
iii) Dry the beads and coat with dispersion of ingredients 6 and 7 in 8.
iv) Blend the 3 different beads together in a ratio of 1:1:1.
v) Compress the material of step (iv) into tablets.

### Example-12

| S. No. | Ingredient | Quantity (mg/capsule) |
|---|---|---|
| 1. | Nimesulide (micronized) | 100.0 |
| 2. | Cetirizine dihydrochloride | 10.0 |
| 3. | Lactose | 206.5 |
| 4. | Polyoxyl 40 hydrogenated castor oil | 2.0 |
| 5. | Hydroxypropylmethyl cellulose | 31.5 |
| 6. | Magnesium stearate | 2.0 |
| 7. | Colloidal silicon dioxide | 2.0 |

Procedure:
i) Granulate the ingredients from 1 to 3 with ingredients 4 and 5 and pass through a sieve of mesh size 40 (BSS).
ii) Blend the materials of step (i) with ingredients with 6 and 7 and fill into capsules.

### Example-13

A) Quick Release Composition

| S. No. | Ingredient | Quantity (mg/tablet) |
|---|---|---|
| 1. | Nimesulide (micronized) | 25.0 |
| 2. | Lactose | 51.5 |
| 3. | Colloidal silicon dioxide | 3.0 |
| 4. | Povidone K-30 | 3.5 |
| 5. | Docusate sodium | 2.0 |
| 6. | Polysorbate 80 | 4.0 |
| 7. | Magnesium stearate | 0.5 |
| 8. | Croscarmellose sodium | 4.0 |
| 9. | Purified water | q.s. (lost in |
| | | processing) |

Procedure:
i) Sift the ingredients 1, 2 and 3 through a sieve of mesh size 30 (BSS).
ii) Blend the materials of step (i).
iii) Disperse ingredients 4, 5 and 6 in 9 to obtain a homogeneous solution.
iv) Granulate the material of step (ii) with the material of step (iii) and dry the granules thus obtained.
v) Sift 7 and 8 through a sieve of mesh size 40 (BSS) and mix.
vi) Blend the material of step (iv) with the material of step (v).

B) Extended Release Composition

| S. No. | Ingredient | Quantity (mg/tablet) |
|---|---|---|
| 1. | Nimesulide | 75.0 |
| 2. | Lactose | 75.0 |
| 3. | Hydroxypropyl methylcellulose | 4.0 |
| 4. | Povidone K-30 | 4.0 |
| 5. | Docusate sodium | 2.5 |
| 6. | Sodium lauryl sulphate | 1.5 |
| 7. | Magnesium stearate | 1.0 |
| 8. | Colloidal silicon dioxide | 1.0 |
| 9. | Isopropyl alcohol | q.s. (lost in |
| | | processing) |

Procedure:
i) Sift the ingredients 1, 2 and 3 through a sieve of mesh size 30 (BSS).
ii) Blend the materials of step (i).
iii) Disperse ingredients 4 and 5 in 9 to obtain a homogeneous solution.
iv) Granulate the material of step (ii) with the material of step (iii) and dry the granules.
v) Sift the ingredients 6, 7 and 8 through a sieve of mesh size 40 (BSS) and mix.
vi) Blend the material of step (iv) with the material of step (v).
   C) Preparation of Bilayer Tablet
i) Compress the material of step (A) (vi) with the material of step (B) (vi) into bilayer tablets using a suitable compression machine.

### Example-14

A) Immediate Release Fraction

| S. No. | Ingredient | Quantity (mg/tablet) |
|---|---|---|
| 1. | Nimesulide (micronized) | 100.0 |
| 2. | Beta-cyclodextrin | 400.0 |
| 3. | Starch | 70.0 |
| 4. | Povidone K-30 | 7.5 |
| 5. | Purified water | q.s. (lost in |
| | | processing) |
| 6. | Croscarmellose sodium | 20.0 |
| 7. | Magnesium stearate | 2.5 |

Procedure:
i) Mix the ingredients 1 and 2, co-mill under specific conditions favoring complexation using ball mill to prepare a complex.
ii) Mix the complex of step (i) with 3 and granulate with a solution of 4 in 5.
iii) Dry the granules of step (ii) and sift through a sieve of mesh size 20 (BSS).
iv) Sift 6 and 7 through a sieve of mesh size 40 and mix.
v) Blend the material of step (iii) with material of step (iv) to obtain lubricated granules.

B) Extended Release Fraction

| S. No. | Ingredient | Quantity (mg/tablet) |
|---|---|---|
| 1. | Nimesulide | 100.0 |
| 2. | Lactose | 200.0 |
| 3. | Hydroxypropyl methylcellulose | 23.0 |
| | (high viscosity grade) | |
| 4. | Hydroxypropyl methylcellulose | 100.0 |
| | (low viscosity grade) | |
| 5. | Povidone K-30 | 9.0 |
| 6. | Docusate sodium | 4.5 |
| 7. | Purified water | q.s. (lost in processing) |
| 8. | Magnesium stearate | 4.5 |
| 9. | Colloidal silicon dioxide | 4.5 |

Procedure:
i) Sift and mix ingredients 1, 2, 3 and 4.
ii) Granulate the material of step (i) with a solution of 5 and 6 in 7.
iii) Dry the granules of step (ii) and sift through a sieve of mesh size 20 (BSS).
iv) Sift 8 and 9 through a sieve of mesh size 40 and mix.
v) Blend the material of step (iii) with the material of step (iv) to obtain lubricated granules.
   C) Preparation of Tablet
i) Mix the granules obtained in step (A) (v) with the granules obtained in step (B) (v).
ii) Compress the material of step (i) into tablets.

### Example-15

| S. No. | Ingredient | Quantity (mg/tablet) |
|---|---|---|
| 1. | Nimesulide | 200.0 |
| 2. | Hydrogenated vegetable oil | 45.0 |
| 3. | Carbomer | 50.0 |
| 4. | Dibutyl sebacate | 10.0 |
| 5. | Isopropyl alcohol | q.s. (lost in processing) |

Procedure:
i) Melt 2 and granulate 1 with it. Pass the granules through a sieve of mesh size 60 (BSS).
ii) Disperse 3 and 4 in 5.
iii) Coat granules of step (i) with the solution of step (ii) in fluidized bed coater.
iv) Compress the material of step (iii) into tablets.

### Example-16

| S. No. | Ingredient | mg/capsule |
|---|---|---|
| 1. | Nimesulide | 200.0 |
| 2. | Hydroxypropyl methylcellulose | 20.0 |
| | (high viscosity grade) | |
| 3. | Ethyl cellulose | 50.0 |
| 4. | Triethyl citrate | 12.5 |
| 5. | Carbomer | 50.0 |
| 6. | Magnesium stearate | 1.5 |
| 7. | Isopropyl alcohol/Dichloromethane (1:2) | q.s. |
| 8. | Purified water | q.s. |

Procedure:
i) Granulate 1 with an aqueous dispersion of 2. Dry the granules and sift through a sieve of mesh size 60 (BSS).
ii) Dissolve 3 in 7 and add 4.
iii) Coat the granules of step (i) with solution of step (ii) in Fluidized bed coater.
iv) Apply another coat of 5 dissolved in 8 onto the coated granules of step (iii) in fluidized bed coater followed by drying of the granules thus obtained.
v) Sift 6 through a sieve of mesh size 40 (BSS).
vi) Add the material of step (v) to the material of step (iv) and mix.
vii) Fill the material of step (vi) into suitable size hard gelatin capsules.

### Example-17

A) Immediate Release Granules

| S. No. | Ingredient | Quantity/tablet (mg) |
|---|---|---|
| 1. | Nimesulide | 50.0 |
| 2. | Microcrystalline cellulose | 250.0 |
| 3. | Sodium lauryl sulphate | 1.5 |
| 4. | Purified water | q.s. (lost in processing) |
| 5. | Magnesium stearate | 6.5 |

Procedure:
i) Sift 1 and 2 through a sieve of mesh size 40 (BSS) and mix.
ii) Dissolve 3 in 4 with controlled stirring so as to avoid any foam formation.
iii) Granulate the material of step (i) with the material of step (ii).
iv) Dry the granules and sift through a sieve of mesh size 30 (BSS).
v) Sift 5 through a sieve of mesh size 40 (BSS).
vi) Mix the material of step (v) with the material of step (iv).

B) Extended Release Granules

| S. No. | Ingredient | Quantity/tablet (mg) |
|---|---|---|
| 1. | Nimesulide | 150.0 |
| 2. | Lactose | 100.0 |
| 3. | Microcrystalline cellulose | 100.0 |
| 4. | Sodium lauryl sulphate | 1.5 |
| 5. | Polyvinyl pyrrolidone | 15.0 |
| 6. | Hydroxypropyl methylcellulose | 15.0 |
| 7. | Purified water | q.s. (lost in processing) |
| 8. | Magnesium stearate | 6.5 |

Procedure:
i) Sift 1, 2 and 3 through a sieve of mesh size 30 (BSS) and mix.
ii) Dissolve 4 and 5 in 7 with controlled stirring so as to avoid any foam formation.
iii) Granulate the material of step (i) with the material of step (ii).
iv) Dry the granules and sift through a sieve of mesh size 20 (BSS).
v) Sift 6 and 8 through a sieve of mesh size 40 (BSS).
vi) Mix the material of step (v) with the material of step (iv).
   C) Preparation of Inlay Tablet

Compress the material of step (A) (vi) to form the outer layer on the material of step (B) (vi), producing inlay tablets.

### Example-18

| S. No. | Ingredient | Quantity/tablet (mg) |
|---|---|---|
| 1. | Nimesulide granules prepared in | 200.0 |
| | Example-18, step (B) (vi) equivalent to | |
| | Nimesulide | |
| 2. | Sodium bicarbonate | 400.0 |
| 3. | Polyethylene glycol 6000 | 20.0 |
| 4. | Magnesium stearate | 10.0 |
| 5. | Purified talc | 10.0 |

Procedure:
i) Mix 1, 2, 3, 4 and 5 after sifting through a sieve of mesh size 30 (BSS).
ii) Compress the material of step (i) into tablets.

### Example-19

| S. No. | Ingredient | Quantity/tablet (mg) |
|---|---|---|
| 1. | Non-Pareil beads | 350.0 |
| 2. | Nimesulide | 200.0 |
| 3. | Lactose | 30.0 |
| 4. | Ethyl cellulose | 350.0 |
| 5. | Diethyl phthalate | 5.0 |
| 6. | Carbomer | 50.0 |

Procedure:
i) Coat 1 with 2, 3 and 4 using 5 as the plasticizer.
ii) Disperse 6 in Isopropyl Alcohol/Water and coat the drug loaded beads of step (i) with above solution in Fluidized Bed Coater.
iii) Compress the material of step (ii) into tablets.

### Example-20

A) Immediate Release Fraction

| S. No. | Ingredient | Quantity (mg/tablet) |
|---|---|---|
| 1. | Nimesulide (micronized) | 50.0 |
| 2. | Hydroxypropyl methylcellulose | 40.0 |
| 3. | Hydroxyethyl cellulose | 10.0 |
| 4. | Lactose | 100.0 |
| 5. | Povidone | 5.0 |
| 6. | Purified water | q.s. (lost in processing) |
| 7. | Magnesium stearate | 5.0 |

Procedure:
i) Sift 1, 2, 3 and 4 through a sieve of mesh size 40 and mix.
ii) Dissolve 5 in 6.
iii) Granulate blend of step (i) with solution of step (ii) and dry the granules.
iv) Pass the granules through a sieve of mesh size 20.
v) Sift 7 through a sieve of mesh size 40.
vi) Lubricate the granules of step (iv) with the material of step (v).

B) Sustained Release Fraction

| S. No. | Ingredient | Quantity (mg/tablet) |
|---|---|---|
| 1. | Nimesulide (micronized) | 100.0 |
| 2. | Polycarbophil | 75.0 |
| 3. | Lactose | 150.0 |
| 4. | Hydroxypropyl methylcellulose | 20.0 |
| 5. | Magnesium stearate | 2.5 |
| 6. | Isopropyl alcohol | q.s. (lost in processing) |
| 7. | Dichloromethane | q.s. (lost in processing) |

Procedure:
i) Sift 1, 2 and 3 through a sieve of mesh size 40 and mix.
ii) Dissolve 4 in a mixture of 6 and 7.
iii) Granulate the blend of step (i) with the material of step (ii).
iv) Dry the granules and pass through a sieve of mesh size 20.
v) Sift 5 through a sieve of mesh size 40 (BSS).
vi) Lubricate the material of step (iv) with the material of step (v).
   C) Preparation of Tablet

Mix the material of step (A) (vi) with the material of step (B) (vi) and compress the material into tablets.

### Example-21

A) Immediate Release Fraction

| S. No. | Ingredient | Quantity (mg/tablet) |
|---|---|---|
| 1. | Nimesulide | 50.0 |
| 2. | Pregelatinized starch | 46.0 |
| 3. | Hydroxypropyl methylcellulose | 40.0 |
| 4. | Croscarmellose sodium | 5.0 |
| 5. | Polyvinyl pyrrolidone | 4.0 |
| 6. | Purified water | q.s. (lost in processing) |
| 7. | Hydrogenated vegetable oil | 5.0 |

Procedure:
i) Sift 1, 2, 3 and 4 through a sieve of mesh size 40 and mix.
ii) Dissolve 5 in 6.
iii) Granulate blend of step (i) with solution of step (ii) and dry the granules.
iv) Pass the granules through a sieve of mesh size 20.
v) Sift 7 through a sieve of mesh size 40.
vi) Lubricate the granules of step (iv) with the material of step (v).

B) Extended Release Fraction

| S. No. | Ingredient | Quantity (mg/tablet) |
|---|---|---|
| 1. | Nimesulide | 200.0 |
| 2. | Carbomer | 25.0 |
| 3. | Hydroxypropyl cellulose | 50.0 |
| 4. | Xanthan gum | 20.0 |
| 5. | Sodium alginate | 15.0 |
| 6. | Sodium bicarbonate | 100.0 |
| 7. | Lactose | 100.0 |
| 8. | Povidone | 10.0 |
| 9. | Magnesium stearate | 5.0 |
| 10. | Colloidal silicon dioxide | 2.5 |
| 11. | Talc | 2.5 |
| 12. | Isopropyl alcohol | q.s. (lost in |
| | | processing) |

Procedure:
i) Sift 1, 2, 3, 4, 5, 6 and 7 through a sieve of mesh size 40 and mix.
ii) Dissolve 8 in 12 and granulate blend of step (i).
iii) Dry the granules and pass through a sieve of mesh size 20.
iv) Lubricate with 9, 10 and 11.
   C) Preparation of Tablet
i) Mix the material of step (A) (vi) with the material of step (B) (iv).
ii) Compress the material of step (i) into tablets.

### Example-22

| S. No. | Ingredient | Quantity (mg/tablet) |
|---|---|---|
| 1. | Nimesulide (micronized) | 200.0 |
| 2. | Lactose | 100.0 |
| 3. | Hydroxypropylmethyl cellulose | 50.0 |
| 4. | Polycarbophil | 50.0 |
| 5. | Povidone/Vinyl acetate copolymer | 10.0 |
| 6. | Magnesium stearate | 5.0 |
| 7. | Talc | 5.0 |
| 8. | Methacrylic acid copolymer | 33.0 |
| 9. | Triethyl citrate | 7.0 |
| 10. | Talc | 5.0 |
| 11. | Isopropyl alcohol | q.s. (lost in |
| | | processing) |
| 12. | Dichloromethane | q.s. (lost in |
| | | processing) |

Procedure:
i) Sift 1, 2, 3, and 4 through a sieve of mesh size 40 and mix.
ii) Dissolve 5 in 12 and granulate with the blend of step (i).
iii) Dry the granules and pass through a sieve of mesh size 20.
iv) Lubricate the granules with 6 and 7.
v) Compress the material of step (iv) into tablets.
vi) Dissolve 8 and 9 in a mixture of 11 and 12.
vii) Disperse 10 in the solution of step (vi).
viii) Coat the tablets of step (v) with the material of step (vii).

### Example-23

| S. No. | Ingredient | Quantity (mg/tablet) |
|---|---|---|
| 1. | Nimesulide (micronized) | 200.0 |
| 2. | Lactose | 100.0 |
| 3. | Polyethylene oxide | 50.0 |
| 4. | Polyvinyl alcohol | 25.0 |
| 5. | Polyvinyl acetate | 20.0 |
| 6. | Isopropyl alcohol | q.s. (lost in |
| | | processing) |
| 7. | Magnesium stearate | 2.5 |
| 8. | Colloidal silicon dioxide | 2.5 |

Procedure:
i) Sift 1, 2, 3 and 4 through a sieve of mesh size 30 (BSS) and blend.
ii) Dissolve 5 in 6 and granulate the blend of step (i).
iii) Dry the granules and pass through a sieve of mesh size 20 (BSS).
iv) Lubricate with 7 and 8 and compress into tablets.

We claim:

1. A controlled release pharmaceutical composition of nimesulide which comprises nimesulide as an active agent from 0.1% to 99% w/w of the composition, one or more release controlling materials from 0.1% to 99% w/w of the composition and one or more pharmaceutical excipients from 0.9% to 90% w/w of the composition, wherein the said composition is formulated as a gastroretentive system such that the residence time of nimesulide is increased in the stomach, duodenum, jejunum and/or ileum.

2. A controlled release pharmaceutical composition of nimesulide according to claim 1, which is formulated as a compressed or compacted dosage form.

3. A controlled release pharmaceutical composition of nimesulide according to claims 1 or 2, which comprises nimesulide as an active agent from 5% to 95% w/w of the composition, one or more release controlling materials from 2% to 95% w/w of the composition and one or more pharmaceutical excipients from 3% to 80% w/w of the composition.

4. A controlled release pharmaceutical composition of nimesulide as claimed in any of the claims 1-3, wherein the release controlling material is selected from a group comprising cellulose and cellulose derivatives, waxes, carbomers, polyalkylene polyols, polycarbophils, methacrylic acid derivatives, gelatins, gums, polyethylene oxides, and polyvinyl pyrrolidone, or mixtures thereof.

5. The composition as claimed in any of the claims 1-3, which further comprises one or more release modifiers selected from a group comprising wetting agents, solubilizers, surfactants, plasticizers, pore formers, pH modifiers or tonicity adjusting agents.

6. A composition as claimed in claim 1, wherein the gastroretention of nimesulide is achieved by mucoadhesion, floatation and/or reducing gastrointestinal motility.

7. A composition as claimed in claim 6, wherein mucoadhesion is achieved by treating nimesulide with polymers having affinity for gastrointestinal mucosa selected from a group comprising polycarbophils, carbomers, alginates, cellulose and cellulose derivatives, chitosan, gums, lectins, or mixtures thereof.

8. A composition as claimed in claim 6, wherein floatation is achieved by adding to the composition one or more gas-generating materials comprising sodium bicarbonate, sodium carbonate, calcium carbonate or potassium carbonate alone or in combination with one or more acidic substances comprising hydrochloric acid, citric acid, fumaric acid, malic acid, maleic acid, ascorbic acid or tartaric acid, or mixtures thereof.

9. A composition as claimed in claim 6, wherein gastrointestinal motility is reduced by adding materials selected from a group comprising fats, fatty acids and transesterification products of fats or fatty acids with polyols, or mixtures thereof.

10. A composition as claimed in claim 1, wherein the other pharmaceutically acceptable excipients are selected from a group comprising of diluents, binders, disintegrants, colorants, lubricants, antiadherants, plasticizers, coating agents, opacifiers, antioxidants, stabilizers, preservatives, surfactants, hydrophilic polymers, solubility enhancing agents, osmotic agents, and the like used either alone or in combination thereof.

11. A composition according to claim 1, comprising at least two fractions wherein one fraction is an immediate release or fast release fraction providing an immediate release of the active agent and the other fraction is an extended release fraction that releases the active agent over extended periods of time.

12. A composition as claimed in claim 1, wherein the composition is in the form of tablets or capsules such as compressed or compacted dosage forms like tablets or minitablets and hard gelatin capsule or soft gelatin capsule or tablet filled in capsule.

13. A composition according to claim 12, wherein the said composition is formulated as powder, granules, pellets, beads, minitablets, tablets, compacts, shear form particles, floss, flakes, or the like, or combinations thereof.

14. A composition as claimed in claim 12, wherein the composition is filled into a capsule or made into a capsule.

15. A composition as claimed in claims 12-14, wherein the composition is formulated as a film coated or enteric coated dosage form.

16. A composition according to claim 12, wherein the tablet or tablet filled in capsule is formulated as multilayer composition.

17. A composition according to claim 12, wherein the tablet or tablet filled in capsule is formulated as bilayered composition, comprising one layer as an immediate release or fast release layer providing an immediate release of the active agent and the other layer as an extended release layer that release the active agent over extended periods of time.

18. A composition according to claim 1, wherein the composition is formulated as a matrix type controlled release dosage form or as an extended release membrane diffusion controlled dosage form or as a site targeted device.

19. A composition according to claim 1, wherein the composition is formulated as an osmotically controlled constant release type device or as pH dependent delayed release type or a pulsatile release type dosage form or as hydrodynamically balanced system.

20. A composition according to claim 1, wherein the composition is formulated in a bimodal release form such as an immediate release form to provide an initial loading dose of the active agent and a delayed release form to provide a dose of the active agent with a lag time for an extended duration.

21. A composition according to claim 1, wherein the composition is formulated as effervescent or dispersible system.

22. A composition according to claim 12, wherein the composition is formulated as gastric mucoadhesive controlled release microspheres or as mucoadhesive monolithic or multilayered tablets.

23. A composition according to any of the claims 1-22, wherein the composition additionally comprises a permeation enhancer.

24. A composition according to claim 23, wherein the permeation enhancer is selected from a group comprising Vitamin E tocopheryl propylene glycol succinate, piperine, a lipid, a surfactant, or mixtures thereof.

25. A composition according to claim 1, wherein the composition is in the form of a multiparticulate composition comprising a blend of one or more types of particles, granules, pellets, beads, compacts, shear form particles, floss, or the like, or combinations thereof, having different release characteristics.

26. A composition according to claim 25, wherein the multiparticulate composition is in the form of a compressed or compacted minitablet or tablet or a hard gelatin capsule or a soft gelatin capsule.

27. A composition according to claim 1, wherein the controlled release composition is in the sustained release form, timed release form, pulsatile release form, prolonged release form, extended release form or delayed release form, or a combination thereof.

28. A composition according to claim 27, wherein the controlled release form is in the form of a combination of immediate release form and extended release form.

29. A composition according to any of the claims 1-28, which additionally comprises one or more other active agent(s).

30. A composition according to claim 29, wherein the additional active agent(s) is selected from a group comprising antihistaminics, antispasmodics, antipyretics, and the like or mixtures thereof.

31. A process for the manufacture of controlled release pharmaceutical composition of nimesulide according to claim 1, which comprises of the following steps:
i) treating the active agent nimesulide in an amount of from 0.1% to 99% w/w of the composition, with one or more release controlling materials in an amount of from 0.1% to 99% w/w of the composition,
ii) optionally adding one or more pharmaceutical excipients in an amount of from 0.9% to 90% w/w of the composition,
iii) formulating the material of step (ii) into a suitable composition.

32. A method of treatment of NSAID indicated disorder(s) which comprises administrating to a patient in need thereof a pharmaceutically effective amount of the composition according to claim 1.

33. A method of treatment according to claim 32, wherein the NSAID indicated disorder(s) is selected from a group comprising pain and/or inflammation associated with osteoarthritis; dental extraction or surgery; saphenectomy or inguinal hernioplasty; haemorrhoidectomy; acute musculoskeletal injury; ear, nose or throat disorders; gynaecological disorders; cancer pain; alzheimer's disease; thrombophlebitis; urogenital disorders; bursitis or tendonitis; morning stiffness associated with rheumatoid arthritis, pain associated with fever and/or any inflammation, and the like, or a combination thereof.
